(19) United States
(12) Patent Application Publication
(10) Pub. No.: US 2024/0216718 A1
(43) Pub. Date: Jul. 4, 2024

(54) PORTABLE ULTRASOUND SYSTEM AND METHODS OF TREATING FACIAL SKIN BY APPLICATION OF SURFACE ACOUSTIC WAVES

(71) Applicant: NANOVIBRONIX, INC., Elmsford, NY (US)

(72) Inventor: Brian Murphy, Fort Lauderdale, FL (US)

(21) Appl. No.: 17/646,753

(22) Filed: Jan. 3, 2022

(51) Int. Cl. A61N 7/00; B06B 1/06

(52) U.S. Cl. CPC A61N 7/00; B06B 1/0651; A61N 2007/0034; A61N 2007/0078

(57) ABSTRACT

A portable ultrasound system, such as a handheld ultrasound device or a flexible patch, includes an ultrasound transducer in direct contact with a portion of facial skin. The ultrasound transducer generates surface acoustic waves (SAW) across the portion of facial skin to improve properties of the skin and/or enhance absorption of a facial skin care product by enabling an active ingredient of the facial skin care product to penetrate the stratum corneum layer and be delivered to sub-dermal tissues of the skin.

### FIELD

Embodiments of the present disclosure relate generally to methods and devices for creating surface acoustic waves (SAW) on skin, and specifically to cosmetic devices and methods for applying SAW to facial skin to reduce signs of aging and improve texture.

### BACKGROUND OF THE INVENTION

The skin, a full one-sixth of body weight, is a sophisticated and dynamic organ, which serves as a bulwark between the sensitive internal tissues of the body and the external environment. Not a mere barrier, the integument is involved in the maintenance of body temperature and internal hydration, sensory functions, and immunological surveillance.

The skin is incredibly durable, but like all other systems, it eventually succumbs to the inexorable effects of aging. The skin is also the most visible indicator of age. The process of aging affects the skin in multiple ways. The epidermis thins and turnover rate slows dramatically. The skin becomes more fragile and vulnerable to damage and more sensitive to irritating environmental factors and allergens. Collagen cross-linking is damaged and as a result, the skin loses much of its strength and elasticity. The fat content in the skin is decreased, resulting in a less plump and smoother look. The number of blood vessels in the skin decreases, and the skin loses its youthful color and glow. As the moisture holding capacity is decreased, the skin becomes dry and loose.

Mild treatment of the skin by ultrasound is a commonly practiced method used by cosmeticians for cosmetic skincare.

SAW actuated devices and disposable patches can address a host of cosmetic and topical concerns, including moisturizing, firming, anti-cellulite, acne, facial redness, sun damage repair. The use of SAW can enhance the permeation of compounds, due to elliptical motion of particles during micro vibration on the surface, and due to micro-streaming resulting therefrom. Thus, micro-electronic skin care products provide a significant increase in the percentage of active cosmetic ingredients that can be delivered onto the upper layers of the skin.

### SUMMARY OF THE INVENTION

The present invention is directed to cosmetic methods for treating facial skin and underlying layers. Specifically, the present invention can be used to achieve rejuvenation, local improvement of the blood circulation, heating of the tissue, accelerated enzyme activity, and enhancement of natural healing processes.

According to one aspect of the invention there is provided a method for treating human skin. The method includes positioning an actuator on the skin, electrically connecting the actuator to a processor, activating the actuator via the processor, producing surface acoustic waves on the skin based on the activating, and controlling parameters of the activating so as to achieve particular treatment effects on the skin by the produced surface acoustic waves.

According to another aspect of the invention, there is provided a handheld ultrasound device for treatment of skin. The device includes a skin-contacting portion, an actuator incorporated into the skin-contacting portion, the actuator for producing surface acoustic waves on the skin, and a processor for controlling the actuator In yet another aspect of the invention, an actuator is incorporated into one or more flexible patches for application to the face.

Described herein is a portable ultrasound system comprising: an energy generating module operative to generate a driving signal that can be transformed into ultrasonic energy, wherein said energy generating module comprises a power source, an oscillator, and a driver component; and an ultrasound transducer comprising a piezoelectric component, said ultrasound transducer being operative to receive the driving signal from the energy generating module, to transform the driving signal into ultrasonic energy, and to control a direction of the ultrasonic energy emitted from the ultrasound transducer, wherein the oscillator and driver component are housed on or within the ultrasound transducer, and the power source is not housed on or within the ultrasound transducer. In embodiments, the ultrasound energy from the transducer maybe emitted as pulsed, continuous, or both pulsed and continuous ultrasonic energy. In certain embodiments, the energy generating module of the portable ultrasound system comprises a voltage controller operative to control power distribution from the power source to the oscillator and driver component. The voltage controller may comprise an on/off controller coupled to a transistor switch.

In embodiments, the portable ultrasound system further comprises a facial skin care product comprising an active agent for improving a state of the facial skin, and the ultrasound transducer is incorporated into a skin-facing surface of a flexible patch configured to be adhered to the facial skin near the portion of skin.

Also described is a handheld ultrasound device, comprising: a housing sized for grasping by a human hand, the housing defined by an elongated handle portion and a head portion extending transversely from an upper end of the handle portion and having a contacting portion interfacing with a subject's skin; an actuator comprising a metallic layer disposed within the contacting portion; and a processor and battery comprised within the handle portion and communicably connected to the actuator, wherein the metallic layer is positioned in an outward direction from the contacting portion so as to come into contact with facial skin during treatment. In preferred embodiments, the metallic layer of the actuator is a piezoelectric plate.

In embodiments, the actuator of a handheld ultrasonic device of the invention may be configured to have multiple function modes, the multiple function modes comprising a micro-massaging action of facial skin interfacing with the contacting portion. In certain embodiments, the handheld ultrasonic device further comprises a detachable massage unit extending from the contacting portion.

Also described herein are methods of treating, improving, and/or rejuvenating a subject's facial skin.

In embodiments, a method of rejuvenating a subject's facial skin, comprises: activating the handheld ultrasound device described herein to begin SAW-generation by the actuator; and manipulating the user's facial skin by placing the contacting portion of the handheld device against the user's facial skin. The method may further comprise applying a cosmetic active agent to the facial skin.

In additional embodiments, provided is a method of treating a subject's facial skin comprising: providing a facial skin care product comprising an active ingredient for improving skin quality to a portion of facial skin; directing acoustic energy in the form of surface acoustic waves (SAW) at the portion of facial skin comprising the facial skin care product, and causing the SAW to propagate along a boundary layer of the facial skin, wherein the SAW enable the active ingredient to penetrate a stratum corneum layer of the skin and be delivered to sub-dermal tissue to improve quality the facial skin. The directing of acoustic energy in the form of SAW may comprise using a handheld ultrasound device to direct the acoustic energy to the portion of the facial skin comprising the facial skin care product, the handheld ultrasound device comprising a handle portion and a head portion extending transversely from an upper end of the handle portion and having a contacting portion interfacing with the portion of facial skin comprising the facial skin care product, the contacting portion comprising a piezo actuator disposed in a skin-contacting surface thereof so as come in direct contact with the facial skin. In embodiments, the SAW are high frequency and low energy elastic waves with a vibration amplitude in a range of from 0.2 to 2 nanometers generated by the piezo actuator. In still further embodiments, directing acoustic energy in the form of SAW using the handheld ultrasound device results in acoustic pressure creating a micro-cavitation effect on the portion of the skin, wherein the micro-cavitation results in a temperature increase that is desirable for the treating of the subject's skin.

According to the methods described herein, the directing of acoustic energy in the form of SAW at the portion of facial skin comprising the facial skin care product may comprise applying a flexible patch to the facial skin at a location corresponding or in close proximity to the portion of facial skin comprising the facial skin care product, the flexible patch having a size ranging from 1-60 cm² and comprising an actuator having a size ranging from 0.5-4 cm², wherein, upon activation, the actuator converts a driving signal into SAW propagating along a boundary layer of the facial skin and the flexible patch. In such embodiments, the flexible patch may comprise a circular actuator or a plurality of actuators provided in a circular arrangement to generate standing waves in a central overlapping portion of the facial skin and provide a focused treatment of acoustic energy on the central portion. In certain embodiments, the flexible patch comprises an absorbing material around its edges to reflect the SAW and cause chaotic dispersion of the SAW on the portion of the facial skin treated by the flexible patch.

### BRIEF DESCRIPTION OF THE DRAWINGS

The above and further advantages of the present invention may be better understood by referring to the following description in conjunction with the accompanying drawings.

It will be appreciated that for simplicity and clarity of illustration, elements shown in the drawings have not necessarily been drawn accurately or to scale. For example, the dimensions of some of the elements may be exaggerated relative to other elements for clarity or several physical components may be included in one functional block or element. Further, where considered appropriate, reference numerals may be repeated among the drawings to indicate corresponding or analogous elements. Moreover, some of the blocks depicted in the drawings may be combined into a single function.

FIG. 1A is a schematic illustration of an actuator for producing surface acoustic waves positioned on an external surface of a portion of skin, in accordance with embodiments of the present invention;

FIG. 1B is a block diagram illustration of a system for treating skin using SAW, in accordance with embodiments of the present invention;

FIG. 2A illustrates elliptical displacement of SAW along a skin surface;

FIG. 2B is a schematic illustration showing the generation of a compressional wave into fluid by a SAW having a wavelength h delivered at an angle e.

FIG. 3 is a cross-sectional illustration of skin with an actuator attached thereto, wherein actuator is comprised of an electromagnetic transducer.

FIG. 4 is an illustration of skin with an actuator where the processor comprises a pulsed laser device.

FIG. 5A is an illustration of an ultrasound transducer according to an embodiment of the invention, wherein the base portion of the ultrasound transducer comprises a piezo-element and acts as the activating portion;

FIG. 5B is an illustration of the ultrasound transducer during vibrations upon activation;

FIG. 6 is a schematic illustration of SAW activity, including depth, intensity and direction.

FIG. 7 is an illustration of a hand-held ultrasound device according to embodiments of the invention.

FIG. 8 is an illustration of a flexible patch for use in embodiments of the invention.

FIG. 9 is an illustration of a flexible patch according to embodiments of the invention.

FIG. 10 is an illustration of a flexible patch having an absorbing material around a perimeter according to embodiments of the invention.

FIGS. 11A-11D are illustrations showing various shapes and configurations of a flexible patch for use in various embodiments of the invention.

### DETAILED DESCRIPTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

In the following detailed description, numerous specific details are set forth in order to provide a thorough understanding of the present invention. It will be understood by those of ordinary skill in the art that the present invention may be practiced without these specific details. In other instances, well-known methods, procedures, components and structures may not have been described in detail so as not to obscure the present invention. Before explaining at least one embodiment of the present invention in detail, it is to be understood that the invention is not limited in its application to the details of construction and the arrangement of the components set forth in the following description or illustrated in the drawings. The invention as described herein is capable of other embodiments or of being practiced or carried out in various ways.

In general, as used throughout the present disclosure, the term “cosmetic treatment” encompasses addressing damage to, repair of, or restoration of soft tissue on the face.

The term “surface acoustic waves” (SAW) as used throughout the present disclosure, includes several types of waves or combinations thereof, as follows: (a) Surface-Rayleigh (elliptical orbit-symmetrical mode); (b) Plate Wave-Lamb-component perpendicular to surface (extensional wave); (c) Plate Wave-Love-parallel to plane layer, perpendicular to wave direction; (d) Stoneley (Leaky Rayleigh Waves)-wave guided along interface; and Sezawa-antisymmetric mode Surface or Rayleigh waves travel along the boundary between two different media, penetrating to a depth of about one wavelength. Lamb waves are a special case of Rayleigh waves, which occur with thin materials.

Conventional ultrasound used for therapy may be of high frequency (1-4 MHz) and low frequency (20-120 KHz), and may have longitudinal or transverse characteristics. The present application discloses excitement of surface acoustic waves on the skin (low-power, low-frequency) and employment of this phenomenon for therapeutic needs. Our theoretical constructions and experimental results proved that low-power, low-frequency ultrasound (20-120 kHz, 0.05-1.0 W/cm²) propagated in the form of surface acoustic waves is effective in one or more of the following: inhibiting adhesion, micro-massage, healing processes, tissue fluid interchange, increased growth of capillary, increased pH of tissue liquids, lowered pain syndrome, resistance of thrombus formation, better drug administering, reduced friction, the cleansing of tissue, the removal of necrotic debris, disinfection, the “biostimulation” of cells, blood flow, micromassaging, drying, intensity of drug diffusion, activeness of the coating agents, and wound healing.

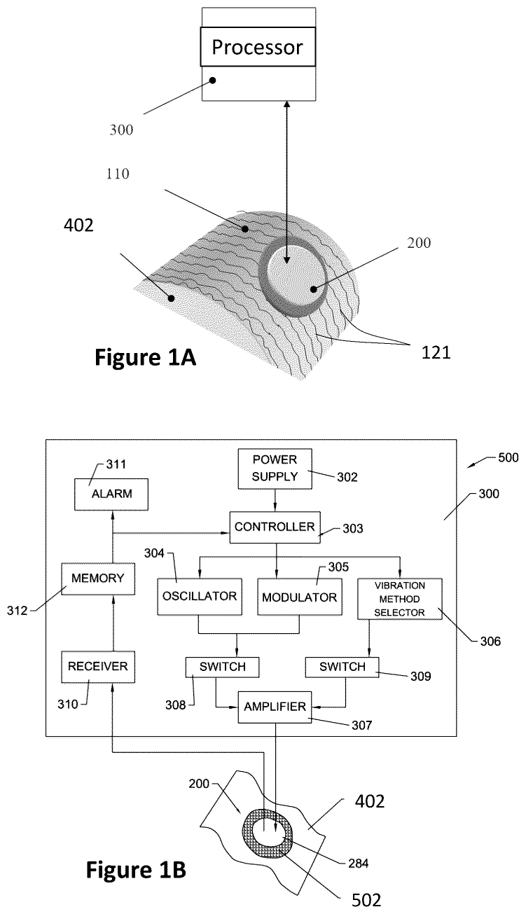

Reference is now made to FIG. 1A, which is a schematic illustration of an actuator 200 for producing surface acoustic waves positioned on an external surface 110 of a portion of skin 402, in accordance with embodiments of the present invention. Actuator 200 is in electrical communication with a processor 300. Processor 300 may be, for example, a central processing unit (CPU), and may include an oscillator, an amplifier, and any other component used for receiving and transmitting signals and making calculations related to the received and transmitted signals. Upon receipt of an electrical signal from processor 300, actuator 200 is capable of generating high frequency mechanical vibrations, in a range from KHz to MHz. These high frequency mechanical vibrations create surface acoustic waves (SAW) 121 (in the nanometer range) on external surface 110 of skin 402. The frequency of generated mechanical oscillations in actuator 200 is directly related to the frequency produced by processor 300. Thus, for example, if oscillations are in the MHz range, the mechanical vibrations will also be in the MHz range, and similarly for other ranges. The energy source applied via processor 300 may have a periodical or non-periodical character, and may be electromechanical, electromagnetic, or electro-optical. Actuator 200 may be comprised of one or multiple piezoelectric transducers, one or more electromagnetic acoustic transducers, or one or multiple laser pulse transducers.

Reference is now made to FIG. 1B, which is a block diagram illustration of a system 500 for treating skin using surface acoustic waves (SAW), in accordance with embodiments of the present invention. The system shown herein is useful in creating SAW via a piezoelectric actuator. However, as noted below, other methods may be used to create SAW as well, including electromagnetic stimulation and laser pulse excitation. System 500 includes an actuator 200, a processor 300 in electrical communication with actuator 200, and optionally a coupler 502 positioned between actuator 200 and skin 402. In the embodiment shown herein, actuator 200 is a piezoelectric actuator, and works by converting electrical signals from processor 300 into mechanical energy, wherein the mechanical energy is transmitted to skin 402, creating on the surface thereof. In some embodiments, actuator 200 is configured to transmit electrical signals proportional to the mechanical energy created to processor 300, and may thus provide a feedback loop to regulate the electrical signals produced by processor 300. Coupler 502 may optionally be placed between actuator 200 and skin 402 in order to match acoustic signal transmission properties of materials of skin 402 and actuator 200.

Processor 300 includes a power supply 302 for providing electrical energy to system 500. In some embodiments, power supply 302 is a separate unit (such as a power cord), and in some embodiments, power supply 302 is incorporated into processor 300 (such as a battery). Processor 300 further includes a controller 303 for controlling output parameters of processor 300. Controller 303 is in electrical communication with an oscillator 304 for providing signals at various frequencies, a modulator 305 for modulating parameters such as frequency, amplitude, etc., and a vibration method selector 306 for providing different types of vibrations, such as single-phase, two-phase or multi-phase vibrations. Oscillator 304 and modulator 305 are connected to a first switch 308, for selection of signal parameters. Vibration method selector 306 is connected to a second switch 309 for selection of vibration method. The selected signal of the selected vibration type is sent through an amplifier 307 to actuator 200. For embodiments wherein electrical signals are sent from actuator 200 to processor 300, these signals are received by a receiver 310 within processor 300. It should be noted that in some instances, signals are sent by a separate sensor placed on or near or incorporated within actuator 200, as will be described in further detail hereinbelow. Signals received by receiver 310 may be sent to a memory module 312, where they are compared with expected values. Results of the comparison are then either sent to controller 303, where signal parameters such as amplitude and frequency may be automatically adjusted based on the received information, or sent to an alarm 311 for alerting a user that parameters should be adjusted.

Selection of parameters depends on the use and application of system 500, and may vary according to specific requirements. For example, when actuator 200 is applied directly to the skin surface, frequencies may be in a range of 0.1 Hz-10 MHz. When an interface is present, such as a cream, drug, or other active ingredient, frequencies may be in a range of 1 KHz-20 KHz so as to provide higher energy waves that can penetrate the interface. Alternatively, higher energy may be accomplished by modulation of waves to produce increased amplitudes. Pulsed or continuous inputs may be used. The types of waves may also differ depending on the type of treatment desired. For example, acne may be treated by focused waves, as will be described in further detail hereinbelow, while micro-massage may be accomplished via a large range of wave types. Microstreaming may also be accomplished via a large range of wave types; however, the speed of microstreaming may vary based on the chosen parameters. Speed of microstreaming may be in a range of 1 nm/minute to 10 microns/minute.

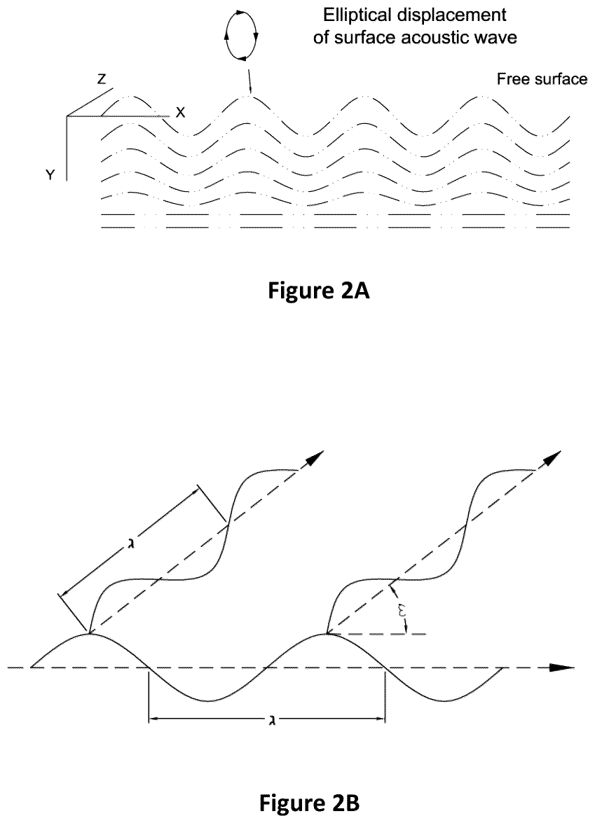

Reference is now made to FIG. 2A, which is a schematic representation (not to scale) showing the propagation of a Rayleigh wave on an elastic surface. As shown in FIG. 2A, the physical motion of this “true-SAW” wave type is associated with mechanically time-dependent elliptical displacement of the surface structure. One component of the physical displacement is parallel to the SAW propagation axis X, and another component is normal to the surface along axis y. In general, the amplitude of surface displacement along the y-axis is larger than along the SAW propagation axis x. The amplitudes of both SAW displacement components are negligible for penetration depths (into the body of the solid, such as, for example skin 402) greater than a few acoustic wavelengths. Propagation of Lamb waves depends on density, elastic, and other material properties of the solid (such as skin 402, for example), and they are influenced a great deal by selected frequency and material thickness. With Lamb waves, a number of modes of particle vibration are possible, but the two most common are symmetrical and antisymmetrical. The complex motion of the particles is similar to the elliptical orbits for surface waves.

Reference is now made to FIG. 2B, which is a schematic illustration showing the generation of a compressional wave into fluid λ₁ by a SAW having a wavelength λ and delivered at an angle 5 to external surface 110 of skin 402. Pressure (gas or fluid loading) also contributes to acoustic wave attenuation and velocity change. In this case, attenuation is due to the generation of compressional waves in the gas or fluid in contact with a surface of skin 402. Thus, the shear vertical component of the wave causes periodic compression and rarefaction of the gas or fluid, resulting in a coupling of acoustic energy from skin 402 into the gas or fluid. The condition for this to occur is: cos angle ξ=λ/λ₁.

The presence of SAW on external surfaces 120 and 110 of skin 402 causes a pushing/pulling effect of materials on these surfaces, including fluids and particulates suspended therein, whereby the SAW may be used to augment tissue therapy. There are several methods for producing SAW on skin, including electromagnetic, laser pulses, or piezoelectric methods, as will be discussed in greater detail hereinbelow.

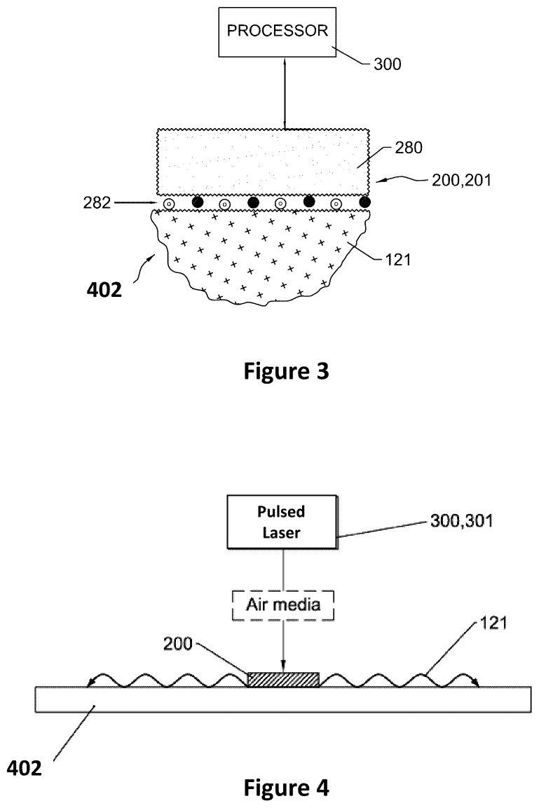

Illustrated in FIG. 3 is a cross-sectional illustration of skin 402 with an actuator 200 attached thereto, wherein actuator 200 is comprised of an electromagnetic transducer 201. As shown in FIG. 3, actuator 200 is comprised of a base portion 280 and an activating portion 282. Base portion 280 may be of any conductive material, such as a metal, and activating portion 282 is comprised of electromagnetic transducers, such as electromagnetic ultrasound transducers available from Olympus company, Panametrics-NDT Ultrasonic Transducer. Base portion 280 may be the face of electromagnetic transducer 201 in embodiments, activating portion 282 may be configured to excite Lamb waves. This type of actuator vibrates the atoms within skin 402. Processor 300 is in electrical communication with base portion 280. Processor 300 applies a current to base portion 280, which is comprised of an electrically conductive material. When the current is applied at a particular ultrasonic frequency, activating portion 282 creates vibrations of Lamb wave type, wherein the distance between max amplitudes will be equal to one-half the wavelength of SAW excited on the skin 402.

Reference is now made to FIG. 4, which is a diagrammatic illustration of skin 402 with a processor 300, wherein processor 300 is a pulsed laser device 301. Actuator 200 is a metallic plate which is configured to vibrate in response to laser pulses from processor 300. No contact is necessary between actuator 200 and processor 200 since laser pulses travel through the air. Pulsed laser device 301 is used to generate SAW 121 in solids by a thermoelastic mechanism, resulting in elastic displacement of a waveform having a wide band. The frequency range of the SAW excited using pulsed lasers has limited bandwidth as only short pulse widths may be excited with pulsed laser device 301 in a solid. The amplitude and the frequency bandwidth of the laser-induced SAW are improved by decreasing the radius of the focused laser spot.

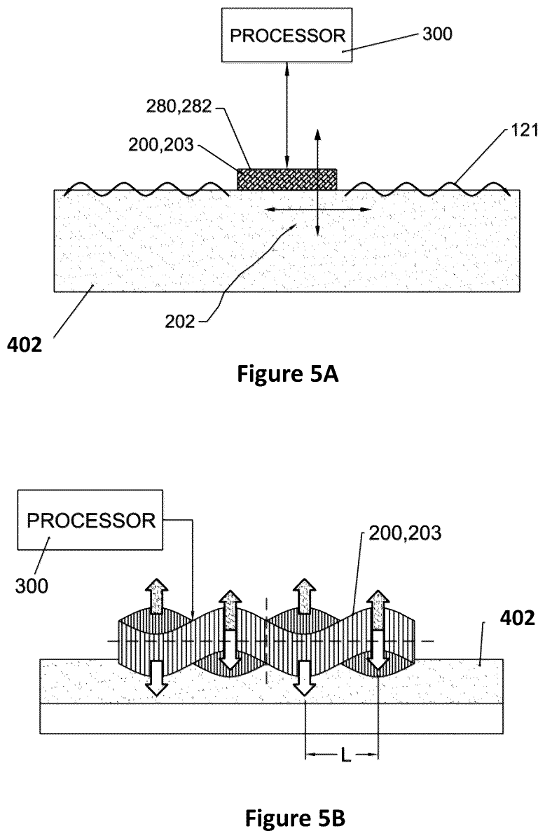

As shown in FIG. 5A, actuator 200 may include one or more piezo-actuators 203, which are configured to provide SAW in accordance with embodiments of the present invention. These piezo-actuators 203 are configured to provide vibrations at amplitudes of between 0.2-2 nanometers. In some embodiments, actuator 200 is comprised of one or multiple piezo-elements 203. Actuator 200 may include a base portion 280 and an activating portion 282, wherein activating portion 282 is comprised of piezo-elements 203. It should be noted that electrodes must be included on piezo-elements 203. In many of the figures, these electrodes are not shown since they may be placed in any location, and the different possibilities for positioning of electrodes are known to those skilled in the art. In some embodiments, base portion 280 is also the activating portion 282 and is thus comprised of piezo-elements 203. When actuator 200 is comprised of a base portion 280 and the base portion 280 is a piezo-element 203, the base portion 280 acts as an activating portion 282. In some embodiments, multiple piezo-elements 203 are used. Actuator 200 may work in thickness and/or radial vibration modes to generate SAW 121 on surfaces of skin 402.

In preferred embodiments, vibrations of piezo-element 203 occur in two planes FIG. 5B illustrates an actuator 200, such as the actuator shown in FIG. 5A, during vibrations. Actuator 200, after activation by processor 300, begins to vibrate in two directions—up and down—as shown by gray and white arrows, respectively. Vibrations of piezo-element 203 generate SAW on the skin surface, where a distance L between two maximal amplitudes of bending vibration modes are proportional to one-half the length L of the SAW.

According to embodiments of the invention, a concentrated SAW effect may be achieved by strategic placement of actuators. In some embodiments, one or more actuators may be connected to one processor. In other embodiments, separate processors may be used for each of the one or more actuators. In embodiments, where multiple actuators are used, applying two actuators at an angle to each other. In some embodiments, multiple actuators may be used in combination to treat one area. In such embodiments, the standing waves induce greater penetration of active agents into the skin's deeper layers. Specifically, mechanical vibrations activated by actuators enable deeper penetration of active agents (drug, serum, gel, etc.) into layers of the skin.

In embodiments, the SAW and/or combined SAW/conventional ultrasound may be further enhanced by the addition of a laser beam. In some embodiments, the conventional ultrasound is continuous and in other embodiments the conventional ultrasound is pulsed. Moreover, the laser therapy may include a pulsed, scanned or gated laser continuous wave laser or incoherent radiation of ultraviolet therapy. In addition, the SAW process and conventional ultrasound and laser energy also stimulates healthy cell growth to aid in granulation and epithelation of skin tissue Embodiments of the present invention relate to methods and systems for facial skin treatments using a SAW process combined with different energy sources, such as a laser, conventional ultrasound, electric current, magnetic field, ultraviolet, microwaves, radio frequency, light-emitting diodes (LEDs) and or equivalent sources, as will be apparent to one skilled in this art.

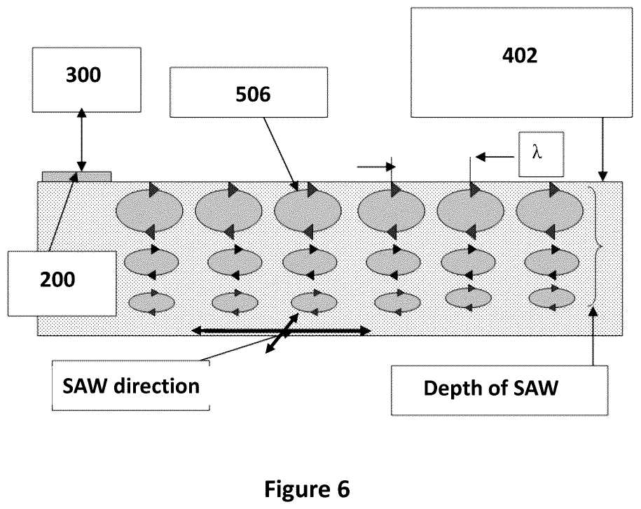

Reference is now made to FIG. 6, which is a schematic illustration of SAW activity, including depth, intensity and direction SAW penetration may be to depths of up to two wavelengths and is largely dependent on frequency. As shown in FIG. 6, when SAW has a relatively long wavelength, deep penetration is achieved. Particles vibrate elliptically, as indicated by ellipses 506, and the energy intensity decreases with increased distance from the surface. The intensity is controlled by the voltage applied by processor 300, and the wavelength is controlled by the frequency applied. SAW actuators 200 may work in a range of between 10 Hz-10 MHz in continuous and pulse regimes. These features enable management of the depth of SAW distribution in a discrete or continuous manner.

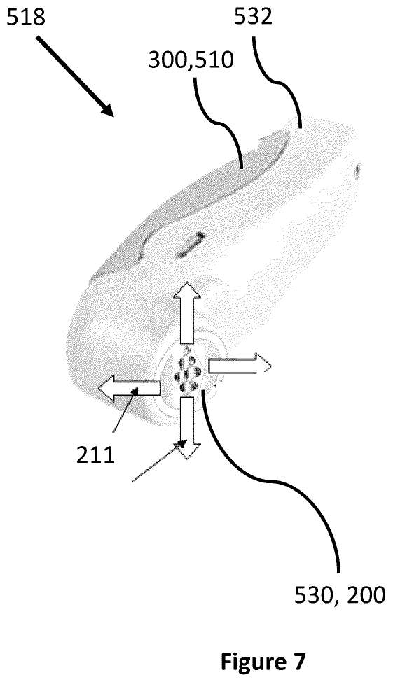

FIG. 7 shows a hand-held ultrasound device 518 according to embodiments of the invention. As shown, the hand-held ultrasound device 518 comprises a contacting portion 530 configured to be placed in direct contact with the facial skin during treatment. Am actuator 200 is preferably comprised of a thin piezo-electric plate and is disposed within the contacting portion 530. Specifically, the actuator 200 is comprised of a metallic layer and a piezoelectric layer with an electrode thereon, wherein the metallic layer is positioned in an outward direction from the contacting portion 530 so as to come into contact with the facial skin during treatment. In embodiments, the hand-held ultrasound device 518 has a handle 532 attached to the contacting portion 530, which may be configured for optimal fit and control with a hand of a user. A processor 300 and a battery 510 may be comprised within the handle 532 and communicably and/or operably connected to an actuator 200. The actuator 200 of the hand-held ultrasound device 518 may have dual (or multiple) function modes, including (without limitation) micro-massaging action of skin under the contacting portion 530, and, as shown by arrows 211, generating a spread of excited surface acoustic waves on the skin round the actuator 200.

When the hand-held ultrasound device is in an active state, the following energy parameters may be observed on a surface of the contacting portion 530 beyond/around the actuator 200: Spatial average, temporal average intensity. I_{SATA}=10 mW/Cm²; Spatial peak, temporal average intensity. I_{SPTA}=:55 mW/cm². As explained herein, the actuator 200 produces SAW waves on and surrounding the contacting portion 530 of the device 518. These SAW waves can furthermore be transmitted at a predetermined depth around the skin-contacting surface that corresponds to a distance A as it relates to the SAW wavelength. The acoustic energy generated by the SAW from the actuator dissipates with distance from the contacting portion 530. The acoustic energy dissipates distancing from the active surface of the contacting portion 530.

In addition to achieving deeper penetration of active agents through transmission of SAW, the hand-held ultrasound device according to the present invention provides a micro-massaging function of facial skin, which further improves oxidation, hydration, and penetration of active agents into the skin.

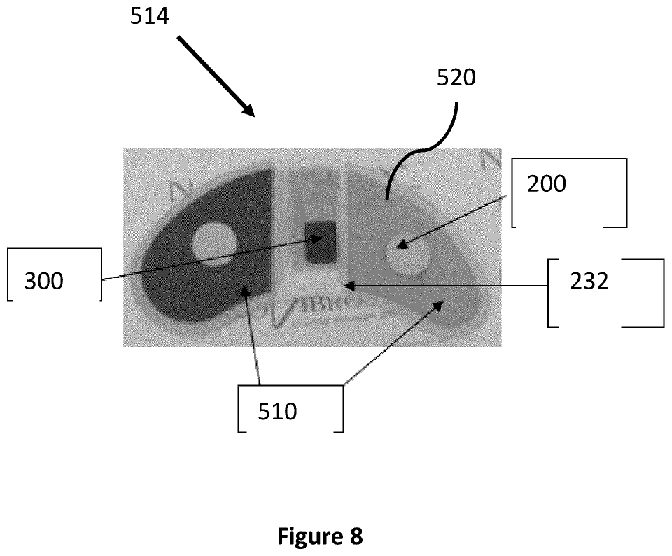

Turning to FIG. 8, provided is a flexible patch 514 or use according to various embodiments of the invention. As shown in FIG. 8, the patch may have an arch-like configuration with two substantially corresponding sides, with each side comprising an actuator 200. In alternative embodiments (not shown), only a single actuator 200 may be included in the patch 514. A sensor 232 may be provided near a central portion of the patch 514. One or more batteries 510, such as one or more thin, flexible batteries available from Power Paper, Nec or Solicore may also be incorporated into the patch 514. An adhesive layer 520 may be provided around the circumferential outer edge of the inner surface (facing the skin) of the patch 514 to adhere the patch 514 to the skin. The patch 514 may also include a removable protective layer (not shown) disposed over the adhesive layer 520, which may be peeled off from the adhesive layer 520 prior to application of the patch 514 to the skin. In some embodiments, the protective layer may be configured as a switch such that, by removing the protective layer from the inner surface of the patch 514, the one or more batteries 510 become activated.

In some embodiments, the piezo-element actuator may be a piezo bender, and may consist of thin piezo material layer glued to a thin metallic plate. The piezo-element is incorporated into the patch base material in such a manner that the metallic plate faces outward with respect to the skin, and the piezo material plus the adhesive layer of the patch face inward with respect to the skin. When activated by processor 300, actuators 200 create standing wave on the metallic surfaces. These standing waves penetrate into the depths of the skin layers, resulting in pain relief effect due to enhanced diathermy effect. Furthermore, the standing wave is a generator of SAW on the areas surrounding the actuator surfaces, which enhance biological processes (blood flow, liquid and gas exchange, and etc.) further resulting in pain relief.

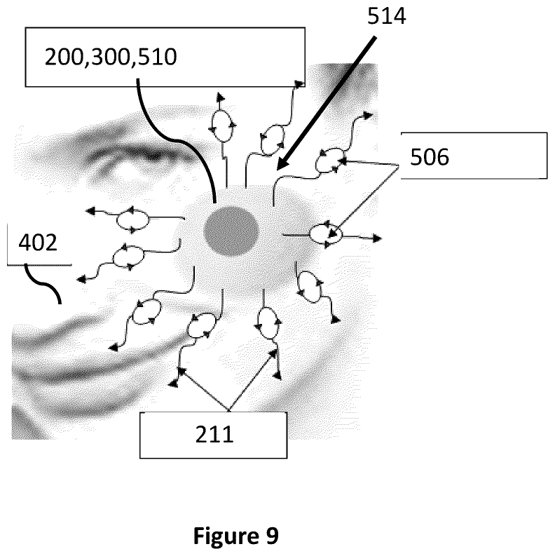

Shown in FIG. 9 is a flexible patch 514 disposed on facial skin 402 according to embodiments of the invention, wherein the patch 514 is configured to continuously distribute SAW waves in various directions as shown by arrows 211 and ellipses 506. In such embodiments, flexible patch 514 includes one or more SAW actuators 200, a processor 300, and at least one battery 510 incorporated therein. The at least one battery 510 may be configured to provide energy in a range of 5-30 mW/cm², and processor 300 may be configured to simultaneously control the one or more actuators 200. Furthermore, processor 300 may be configured to simultaneously control a plurality of patches 514. In embodiments, depth of SAW propagation may be selectively managed by varying the wave frequency. As shown in FIG. 9, the actuator 200 distributes the SAW waves continuously and in various directions. Alternatively, the actuator 200 may distribute SAW waves in only one direction (not shown). Furthermore, the SAW waves may be absorbed and reflected due to energy absorbers such as gauze, synthetic porous materials, gum, and the like, which can prevent or hinder SAW waves from being propagated in other directions.

In some embodiments, flexible patch 514 includes multiple actuators 200, controllers and batteries 510, each of which constitutes an independently-operating system. The propagation and distribution of SAW waves in several directions, as shown in FIG. 9, can result in standing waves in an overlapping area on the skin 402, providing a more focused treatment of acoustic energy applied thereto. In some embodiments, actuator 200 is ring-shaped, or a plurality of actuators 200 may be provided in a circular arrangement. A circular configuration of actuators 200 allows for standing waves to be created in a central portion, thus further concentrating the acoustic pressure to create a micro-cavitation effect. In such embodiments, the micro-cavitation results in temperature increasing to about 70° C., making the ring or circular arrangement of actuators well-suited for treatments where high energy may be desirable, such as, e.g., acne healing.

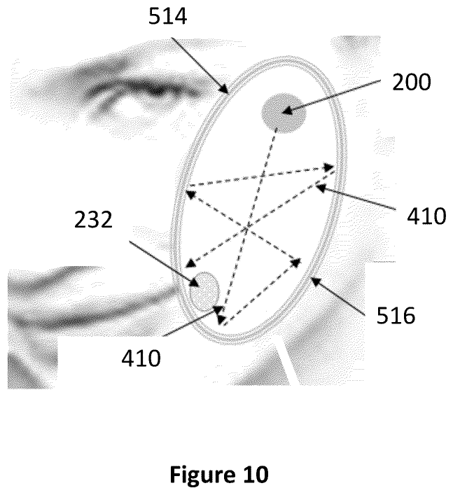

Another illustration of a flexible patch 514 according to embodiments of the invention is provided in FIG. 10. The patch 514 is relatively larger in size than the patch 514 shown in FIG. 9. In such embodiments, the patch 514 may have a size ranging from about 1-60 cm², and comprise an actuator 200 having a size ranging from about 0.5-4 cm². As shown in FIG. 10, flexible patch 514 may further comprise an absorbing material 516 around its edges to reflect the SAW waves. As a result, a chaotic effect of SAW dispersion may be achieved on the area treated by the patch 514, with the chaotic directions of SAW waves being illustrated by arrows 410. In some embodiments, one or more sensors 232 may be incorporated into the flexible patch 514, or placed in close proximity thereto, to measure parameters of the skin, velocity of the SAW waves on the skin, temperature, humidity, and the like.

As discussed further herein, flexible patch 514 may additionally include an active agent, such as a cream, serum, or gel, or an active agent may be separately applied to the facial skin over an area near the flexible patch 514. In such embodiments, SAW waves may be propagated into the active agent to activate chemical components and cause improved or specifically desired distributions thereof. The process may be managed via feedback obtained from the one or more sensors 232. In embodiments where an active agent is incorporated into the patch 514, micro-streaming of SAW waves may result in increased activity of the active agent on the skin and improve the efficacy thereof.

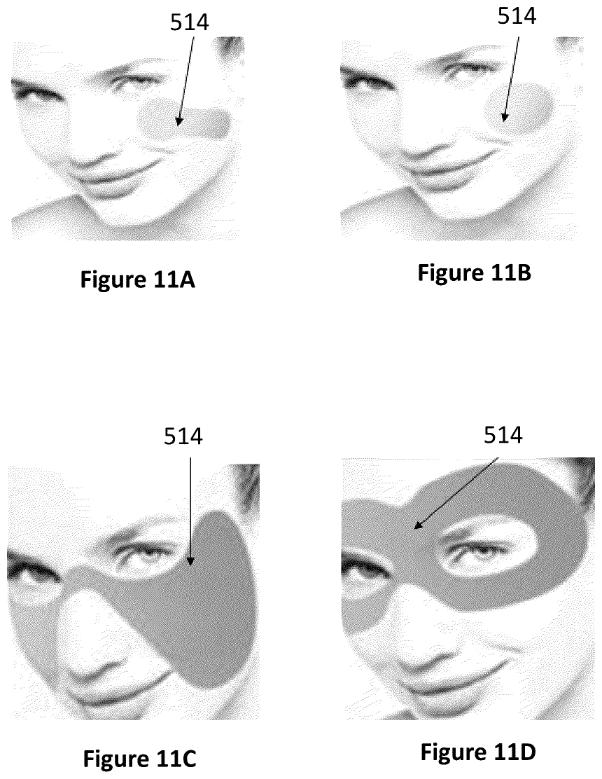

Reference is now made to FIGS. 11A-11D, which are illustrations showing various shapes and configurations of a flexible patch 514 for use in embodiments of the invention. Flexible patch 514 may be elongated, as shown in FIG. 11A, circular, as shown in FIG. 11B, configured to fit on the cheek portions of the face, as depicted in FIG. 11C, configured in an eye-mask configuration, as shown in FIG. 11D, or in any other shape or configuration. Patch 514 may be applied to one or both facial sides and may have a long action time of, e.g., 6 to 8 hours (overnight), or shorter action times in the order of minutes (e.g., 5-20 minutes, or 1 minute) According to embodiments of the invention, the flexible patch described herein may be configured so as to be wirelessly activated and controlled. The acoustic intensity may be regulated depending on the health and location of the facial skin, active agents to be administered (separately or through capillary action of the patch), length of application (active status), and other considerations.

In addition to the main function, actuator may transmit acoustic energy through the patch material to the human skin under the patch. These vibrations cause micro massage at the location of adhesion, thus reducing, and perhaps eliminating, irritation of the skin and making it easier to pull the patch material off after use.

In embodiments of the invention, SAW-activated hand-held ultrasound devices and flexible patches as described herein can enhance delivery of a wide variety of anti-aging, hydrating, and spot whitening molecules to the skin, thus enhancing the power of common cosmetic ingredients, reducing or diminishing wrinkles, decreasing hyper-pigmentation, skin discoloration, sun, and age spots significantly faster than ordinary application of active ingredients alone.

In some embodiments, a flexible patch as described herein may include loaded therein or in a reservoir compartment a drug layer component (liquid, cream or slow-release component of the patch), which may become activated for absorption or dissipation from the reservoir onto the skin upon activation of the patch. Micro streaming and micro pumping due to SAW may result in increased activity of these active components, thus decreasing pain relief time and increasing efficacy. Other possible constructions may have a processor in chip configuration, thus allowing it to be incorporated into the patch. The flexible patches may be configured for repeated use or for one-time application.

According to embodiments of the invention, both thermal and non-thermal mechanisms within the SAW ultrasound field are employed in facial treatments using a flexible patch or a handheld ultrasound device, depending on not just the application mechanism but also the desired cosmetic application. Methods of the invention may additionally reduce the need for needles and more invasive cosmetic procedures typically employed to facilitate delivery of cosmetic agents across the stratum corneum. That is, the stratum corneum (≈10-30 μm) forms a barrier to passive drug diffusion for molecules which have a weight greater than 500 Da. By increasing permeability of the stratum corneum, the methods of the invention facilitate transport of cosmetic agents and protein diffusion across into subcutaneous tissues (dermis). Once an active agent has traversed the stratum corneum, the next layer of skin is typically easier to cross, and the active agent can reach the capillary vessels to be absorbed.

Methods of the invention comprise applying a flexible patch to desired areas of facial skin according to an optimized regimen taking into account frequency and length of application, depth of desired SAW application, and optional additive effects of active agents that may be concurrently or separately applied. Methods of the invention result in the facial skin feeling silky and smooth for several hours, reducing the appearance of fine lines and wrinkles, diminishing the appearance of crow's feet, and smoothing out and lifting skin around the lips and contours of the eyes. The methods may further erase signs of fatigue, reduce the appearance of fine lines and wrinkles, diminish the appearance of crow's feet, smooth out, lift, and improve firmness of skin around the lips and the eye contour, and deliver extra moisture to the skin. Activated SAW anti-aging skincare devices may be produced as a hand-held ultrasound device and/or as flexible patches, as described herein, as well as in a variety of shapes for different application areas.

While the foregoing invention has been described in some detail for purposes of clarity and understanding, it will be clear to one skilled in the art from a reading of this disclosure that various changes in form and detail can be made without departing from the true scope of the invention.

All techniques and apparatus described above can be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

What is claimed is:

1. A portable ultrasound system comprising:
an energy generating module operative to generate a driving signal that can be transformed into ultrasonic energy, wherein said energy generating module comprises a power source, an oscillator, and a driver component; and
an ultrasound transducer comprising a piezoelectric component, said ultrasound transducer being operative to receive the driving signal from the energy generating module, to transform the driving signal into ultrasonic energy, and to control a direction of the ultrasonic energy emitted from the ultrasound transducer,
wherein the oscillator and driver component are housed on or within the ultrasound transducer, and the power source is not housed on or within the ultrasound transducer.

2. The portable ultrasound system according to claim 1, wherein the ultrasound energy from the transducer is emitted as pulsed, continuous, or both pulsed and continuous ultrasonic energy.

3. The portable ultrasound system according to claim 1, wherein the energy generating module comprises a voltage controller operative to control power distribution from the power source to the oscillator and driver component.

4. The portable ultrasound system according to claim 3, wherein the voltage controller comprises an on/off controller coupled to a transistor switch.

5. The portable ultrasound system according to claim 1, further comprising a facial skin care product comprising an active agent for improving a state of the facial skin, and the ultrasound transducer is incorporated into a skin-facing surface of a flexible patch configured to be adhered to the facial skin near the portion of skin.

6. A handheld ultrasound device, comprising:
a housing sized for grasping by a human hand, the housing defined by an elongated handle portion and a head portion extending transversely from an upper end of the handle portion and having a contacting portion interfacing with a subject's skin;
an actuator comprising a metallic layer disposed within the contacting portion; and
a processor and battery comprised within the handle portion and communicably connected to the actuator,
wherein the metallic layer is positioned in an outward direction from the contacting portion so as to come into contact with facial skin during treatment.

7. The handheld ultrasound device according to claim 6, wherein the metallic layer of the actuator is a piezoelectric plate,
a generating module operative to generate a driving signal that can be transformed into ultrasonic energy, wherein said energy generating module comprises a power source, an oscillator, and a driver component; and
an ultrasound transducer comprising a piezoelectric component, said ultrasound transducer being operative to receive the driving signal from the energy generating module, to transform the driving signal into ultrasonic energy, and to control a direction of the ultrasonic energy emitted from the ultrasound transducer,
wherein the oscillator and driver component are housed on or within the ultrasound transducer, and the power source is not housed on or within the ultrasound transducer.

8. The handheld ultrasound device according to claim 6, wherein the actuator is configured to have multiple function modes, the multiple function modes comprising a micro-massaging action of facial skin interfacing with the contacting portion.

9. The handheld ultrasound device according to claim 8, further comprising a detachable massage unit extending from the contacting portion.

10. A method of rejuvenating a subject's facial skin, comprising:
activating the handheld ultrasound device according to claim 6 to begin SAW-generation by the actuator; and
manipulating the user's facial skin by placing the contacting portion of the handheld device against the user's facial skin.

11. The method according to claim 10, further comprising applying a cosmetic active agent to the facial skin.

12. A method of treating a subject's facial skin, comprising:
providing a facial skin care product comprising an active ingredient for improving skin quality to a portion of facial skin;
directing acoustic energy in the form of surface acoustic waves (SAW) at the portion of facial skin comprising the facial skin care product, causing the SAW to propagate along a boundary layer of the facial skin,
wherein the SAW enable the active ingredient to penetrate a stratum corneum layer of the skin and be delivered to sub-dermal tissue to improve quality the facial skin.

13. The method according to claim 12, wherein directing acoustic energy in the form of SAW comprises using a handheld ultrasound device to direct the acoustic energy to the portion of the facial skin comprising the facial skin care product, the handheld ultrasound device comprising a handle portion and a head portion extending transversely from an upper end of the handle portion and having a contacting portion interfacing with the portion of facial skin comprising the facial skin care product, the contacting portion comprising a piezo actuator disposed in a skin-contacting surface thereof so as come in direct contact with the facial skin.

14. The method according to claim 13, wherein the SAW are high frequency and low energy elastic waves with a vibration amplitude in a range of from 0.2 to 2 nanometers generated by the piezo actuator.

15. The method according to claim 13, wherein directing acoustic energy in the form of SAW using the handheld ultrasound device results in acoustic pressure creating a micro-cavitation effect on the portion of the skin, wherein the micro-cavitation results in a temperature increase that is desirable for the treating of the subject's skin.

16. The method according to claim 12, wherein directing acoustic energy in the form of SAW at the portion of facial skin comprising the facial skin care product comprises applying a flexible patch to the facial skin at a location corresponding or in close proximity to the portion of facial skin comprising the facial skin care product, the flexible patch having a size ranging from 1-60 cm² and comprising an actuator having a size ranging from 0.5-4 cm², wherein, upon activation, the actuator converts a driving signal into SAW propagating along a boundary layer of the facial skin and the flexible patch.

17. The method according to claim 16, wherein the flexible patch comprises a circular actuator actuators provided in a circular arrangement to generate standing waves in a central overlapping portion of the facial skin and provide a focused treatment of acoustic energy on the central portion.

18. The method according to claim 16, wherein the flexible patch comprises an absorbing material around its edges to reflect the SAW and cause chaotic dispersion of the SAW on the portion of the facial skin treated by the flexible patch.
